(19) United States
(12) Patent Application Publication
(10) Pub. No.: US 2014/0099379 A1
(43) Pub. Date: Apr. 10, 2014

(54) BIO-COMPATIBLE NANO AND MICROPARTICLES COATED WITH STABILIZERS FOR PULMONARY APPLICATION

(71) Applicants: Moritz Beck-Broichsitter, Marburg (DE); Tobias Gessler, Wettenberg (DE); Thomas Schmehl, Giessen (DE)

(72) Inventors: Moritz Beck-Broichsitter, Marburg (DE); Tobias Gessler, Wettenberg (DE); Thomas Schmehl, Giessen (DE)

(73) Assignee: JUSTUS-LIEBIG-UNIVERSITAET GIESSEN, Giessen (DE)

(21) Appl. No.: 14/122,180

(22) PCT Filed: May 23, 2012
(86) PCT No.: PCT/EP2012/059584

§ 371 Date: Nov. 25, 2013

(30) Foreign Application Priority Data
May 25, 2011 (EP) 11167515.3

(51) Int. Cl. A61K 9/50; A61K 31/519

(52) U.S. Cl. CPC A61K 9/5089; A61K 9/5026; A61K 31/519
USPC 424/497; 514/252.16

(57) ABSTRACT

The present invention provides stabilizers for the coating of biocompatible nano- and microparticles which prevent aggregation of the particles during preparation, storage as well as before and after nebulization and which are suitable to be utilized for the manufacture of a pharmaceutical preparation for pulmonary application.

Biocompatible nano- and microparticles of this invention have a stabilizer layer thickness ranging from 1 to 200 nm and contain an active substance. Said biocompatible nano- and microparticles of this invention can be synthesized for example using the emulsion method known to the expert with subsequent coating by mixing of uncoated particles with the stabilizer, by chemical vapor deposition, by spraying or by covalent attachment.

The present invention provides biocompatible stabilizer-coated nano- and microparticles which are suitable to be used for pulmonary application of active substances. The coating of said biocompatible nano- and microparticles with a stabilizer results in an improved stability and integrity of the particles during nebulization.

### DESCRIPTION OF THE GENERAL FIELD OF INVENTION

The present invention concerns the fields of internal medicine, pharmacology, nano technology and medical technology.

### STATE OF THE ART

Inhalation is known to the expert in this field as a selective route of application of pulmonary drugs, a route by which undesired systemic side effects can be avoided. The direct application of the active substance to the lung facilitates the targeted treatment of respiratory diseases as for example demonstrated for the prostacyclin-antagonist iloprost (Ventavis®) in the treatment of pulmonary hypertension. The relatively short duration of the pharmacological effect after pulmonary drug deposition however is a major disadvantage of inhalation therapy, which thus requires a high frequency of inhalative drug administrations.

Colloidal carrier materials such as e.g. biocompatible nano- and microparticles are known to be suitable pulmonary drug carrier systems. The direct transport of therapeutic agents included in biocompatible nano- and microparticles to the lung allows for a sustained and controlled drug-release at the desired target site which results in a prolongation of the pharmacological effects.

The preparation method of choice for biocompatible nano- and microparticles primarily depends on the physical-chemical parameters of the respective polymer or lipid to be utilized, as well as on the active substance to be included in these biocompatible nano- and microparticles. The choice of the polymer or lipid is determined by criteria such as biocompatibility and biodegradability. In addition, biocompatible nano- and microparticles have to meet further standards like for example a sufficiently high association of the therapeutic agent with the carrier substance as well as a sufficiently high stability against forces occurring during nebulization. These stringent demands are to a great extent met by nano-particular drug carrier systems composed of biocompatible polymers or lipids. If conventional formulations however are used for this purpose, irreversible aggregation of the biocompatible nano- and microparticles may occur during nebulization which leads to a loss of functionality of the respective drug formulation.

Even though the expert in this field knows that stabilizers may potentially modulate the physico-chemical and biological properties of utilized formulations of biocompatible nano- and microparticles, so far no formulations with stabilizers have been disclosed which are able to improve the stability and integrity of biocompatible nano- and microparticles during nebulization and are therefore suitable to prolong the duration of the controlled drug release.

Summarizing, the state of the art has disadvantages with respect to the stability and integrity of nebulized, inhalable formulations of biocompatible nano- and microparticles.

### Aim

Aim of the present invention is to provide stabilizers for biocompatible nano- and microparticles to achieve an improved stability and integrity of said particles during nebulization of a suspension of said particles in such a way that these are better suited for pulmonary application of active substances in humans.

### Solution of the Aim

This aim is solved according to the present invention by the claims, in particular by provision of stabilizers chosen from the group of non-ionic surfactants, anionic surfactants, cationic surfactants, amphoteric surfactants, phospholipids or the polymers.

The stabilizers have the advantage to be utilizable for a variety of nano- and microparticles, being biocompatible and retarding the drug release from particles in such a way that an active substance contained therein is released at the target site over a prolonged period of time as compared to particles without stabilizer.

The stabilizer of this invention is chosen from the group of non-ionic surfactants, anionic surfactants, cationic surfactants, amphoteric surfactants, phospholipids or the polymers. Non-ionic surfactants are for example, but not exhaustively, PEG-PLGA, poloxamers, tween, span, pluronic. Suitable phospholipids are for example dipalmitoylphosphatidylcholine (DPPC), lecithin, distearoylphosphatidylcholine (DSPC) or dimyristoylphosphatidylcholine (DMPC). Suitable polymers are for example natural polymers, synthetic polymers or semi-synthetic polymers.

Among the natural polymers count for example proteins (e.g. albumin), celluloses, esters and ethers thereof, amylose, amylopectin, chitin, chitosan, collagen, gelatine, glycogen, polyamino acids (e.g. polylysine), starch, dextrans or heparins.

Synthetic polymers are for example polyethylene glycol (PEG), polyethyleneimine (PEI), polyvinyl alcohol (PVA), polyvinyl acetate, polyvinyl butyral, polyvinylpyrrolidone (PVP), polyacrylate, poloxamers and diblock or triblock copolymers from PEG and polyester (PLGA, PCL, PLA) (e.g. PEG-PLGA). Semi-synthetic polymers are for example modified starches (e.g. HES).

In a preferred embodiment, coated biocompatible nano- and microparticles of this invention contain polyvinyl alcohol, hereinafter abbreviated as PVA, as stabilizer. PVA is a crystalline, water-soluble plastic.

The stabilizer is particularly suitable to nebulize said coated particles preferably with piezo-electric, jet-, ultrasonic aerosol generators, soft-mist inhalers or metered dose inhalers, i.e. the delivery to the lung is performed via inhalation of an aerosol by means of a nebulizer.

Stabilizers are utilized for the preparation of coated biocompatible nano- and microparticles, for example using the emulsion technique with subsequent solvent evaporation. The thin protective stabilizer layers formed in this process on coated biocompatible nano- and microparticles of this invention consist of surfactants, phospholipids or polymers like for example polyvinyl alcohol (PVA) and improve the stability of said particles during nebulization. The suspension of coated biocompatible nano- and microparticles of this invention is converted into an aerosol suitable for deposition in the respiratory parts of the lung. The characteristic features of coated biocompatible nano- and microparticles of this invention are not influenced by the nebulization procedure. Due to the stabilizer, the prolonged drug release which can be achieved with this new pulmonary drug delivery system for pulmonary active substances results in a reduced frequency of required drug administrations as compared to hitherto used pharmaceutical agents, thus improving both life's quality and therapeutic compliance of the patients.

Exemplarily, stabilizers are used for the stabilization of biocompatible nano- and microparticles. These are advantageously composed of a water-insoluble biocompatible polymer or a water-insoluble lipid as well as a stabilizer and an active substance suitable for pulmonary application. Coated biocompatible nano- and microparticles of this invention are advantageously biodegradable.

The biocompatible polymer is for example a polyester, polyanhydride, polyorthoester, polyphosphoester, polycarbonate, polyketal, polyurea, polyurethane, block copolymer (PEG-PLGA), star polymer or comb polymer.

The polyester is preferred a linear poly(lactic-co-glycolide) copolymer (PLGA-copolymer).

Suitable PLGA-copolymers exist for the preparation of coated biocompatible nano- and microparticles which are utilized for the controlled release of drugs. These include for example, but not exhaustively, copolymers of the Resomer® family. In a preferred embodiment of this invention, the biocompatible nano- and microparticles comprise one of the following Resomer®-substances Resomer® Condensate RG 50:50 Mₙ 2300, Resomer® R2025, Resomer® R202H, Resomer® R2035, Resomer® R203H, Resomer® R2075, Resomer® RG502H, Resomer® RG503H, Resomer®RG504H, Resomer® RG502, Resomer® RG503, Resomer® RG504, Resomer® RG653H, Resomer® RG752H, Resomer® RG752S, Resomer® RG753S, Resomer® RG755S, Resomer® RG756S or Resomer® RG858S. In a particularly preferred embodiment, the coated biocompatible nano- and microparticles of this invention comprise the PLGA-copolymer Resomer® RG502H.

The water-insoluble lipid is for example chosen from the group of acylglycerols (mono, di- or triglycerols). Among the acylglycerols counts for example glycerol monopalmitate.

As active substances are advantageous employed substances chosen from the group of appetite suppressants/antiadipose agents, acidosis therapeutics, analeptics/antihypoxaemic agents, analgesics, antirheumatics, anthelmintics, antiallergics, antianemics, antiarrhythmics, antibiotics, antiinfectives, antidementives, antidiabetics, antidotes, antiemetics, antivertigo agents, antiepileptics, antihemorrhagic agents, haemostatics, antihypertensives, antihypoglycemics, antihypotensives, anticoagulants, antimycotics, antiparasitic agents, antiphogisitics, antitussives, expectorants, antiarteriosclerotics, beta-receptor blockers, calcium channel blockers, inhibitors of the renin-angiotensin-aldosterone system, broncholytics, anti-asthma agents, cholagogics, bile duct therapeutics, cholinergics, corticoids, diagnostics and agents for diagnostic preliminaries, diuretics, circulation-promoting agents, anti-addiction agents, enzyme inhibitors, enzyme-activating or stimulating agents, enzyme deficiency correcting compounds, receptor antagonists, transport proteins, fibrinolytics, geriatric agents, gout agents, influenza drugs, colds and flu remedies, gynecologic agents, hepatics, hypnotics, sedatives, pituitary and hypothalamus hormones, regulatory peptides, hormones, peptide inhibitors, immunomodulators, cardiacs, coronary agents, laxants, lipid-reducing agents, local anaesthetics, neural therapeutic agents, gastric agents, migraine agents, mineral preparations, muscle relaxants, narcotics, neurotropic agents, osteoporosis remedies, calcium/calcium metabolism regulators, remedies for Parkinson's disease, psychopharmaceuticals, sinusitis agents, roborantia, thyroid therapeutics, serums, immunoglobulins, vaccines, antibodies, sexual hormones and their inhibitors, spasmolytics, anticholinergic agents, thrombocyte aggregation inhibitors, antituberculosis agents, urological agents, vein therapeutics, vitamins, cytostatics, antineoplastic agents, homeopathic remedies, vasoactive agents, gene therapeutics (DNA/RNA derivatives), transcription inhibitors, virostatic agents, nicotine, agents against erectile dysfunction, nitric oxide and/or nitric oxide-liberating substances.

In the sense of the present invention also such particles are included as active substances which are for example utilized in diagnostic imaging techniques, but also for therapeutic purposes, e.g. in chemo- and radiotherapy and in hyperthermia therapy.

The term diagnostics includes in vitro as well as also in vivo diagnostic agents. A diagnostic agent to be utilized according to the present invention is for example image-producing and/or radioactive and/or a contrast agent.

Of particular advantage is, due to an increased stability, the utilization of coated biocompatible nano- and microparticles of this invention for the preparation of a pharmaceutical agent for prevention, diagnosis and/or treatment of diseases of the alveolar space as well as for the treatment of respiratory diseases and the utilization of said coated biocompatible nano- and microparticles for the preparation of a pharmaceutical agent suitable for prevention, diagnosis and/or treatment of pulmonary hypertension.

Coated biocompatible nano- and microparticles or this invention can thus be utilized for the preparation of pharmaceutical agents for the treatment and diagnosis of the following diseases: Inflammatory (infectious, non-infectious) and hyperproliferative (neoplastic, non-neoplastic) diseases of the lung and the respiratory tract such as bronchitis, COPD, asthma, pneumonia, tuberculosis, pulmonary hypertension, lung tumors, fibrotic lung diseases, furthermore lung metastases, cystic fibrosis, sarcoidosis, aspergillosis, bronchiectasis, ALI, IRDS, ARDS, alveolar proteinosis, immunosuppression and prophylaxis against infection after lung transplantation.

Conceivable is also a utilization in the case of sepsis, disorders of fat metabolism, tumor diseases, leukemias, innate metabolic disorders (e.g. growth disorders, storage disorders, disorders of the iron metabolism), endocrine diseases for example of the pituitary or the thyroid (Glandula thyreoidea), diabetes, obesity, psychological disorders (e.g. schizophrenia, depression, bipolar affective disorders, posttraumatic stress syndrome, anxiety and panic disorders), CNS disorders (for example M. Parkinson, multiple sclerosis, epilepsy), infectious diseases, rheumatic diseases, allergic and autoimmune diseases, erectile dysfunctions, cardiovascular diseases (for example arterial hypertension, coronary heart diseases, cardiac arrhythmias, heart failure, thromboses and embolisms), renal failure, anaemias, antibody deficiencies, innate or acquired coagulation disorders, platelet function disorders or vitamin deficiency syndromes.

Characterization of Coated Biocompatible Nano- and Microparticles of this Invention

The coated biocompatible nano- and microparticles or this invention have a mean geometric diameter between 10 nm and 10 μm to be easily aerosolizable, and a stabilizer layer thickness between 1 and 200 nm. The stabilizer layer thickness however does not exceed the mean geometric radius of uncoated biocompatible nano- and microparticles. In a preferred embodiment, coated biocompatible nano- and microparticles have a mean geometric diameter between 500 nm and 5 μm to allow a longer-lasting drug release, or between 50 nm and 250 nm to prevent uptake of said particles into macrophages.

Said coated biocompatible nano- and microparticles contain between 0 and 50 (w/w) and in a preferred embodiment between 1 and 20% (w/w) of an active substance suitable for pulmonary application.

Coated biocompatible nano- and microparticles of this invention are preferably nebulized with piezo-electric, air-jet or ultrasonic nebulizers, soft-mist inhalers or metered dose inhalers, i.e. the administration to the lung is performed via inhalation of an aerosol using a nebulizer. The diameter of coated biocompatible nano- and microparticles of this invention is particularly advantageous for a nebulization with herein mentioned nebulizers to achieve a delivery into the depth of the lung. A further route of administration to the lung is via instillation, for example using a catheter, a bronchoscope or a respiratory therapy device (e.g. tube or tracheal cannula). In addition, said coated biocompatible nano- and microparticles can be used for the preparation of a pharmaceutical formulation for prevention, diagnosis and/or treatment of diseases.

### Coating of Biocompatible Nano- and Microparticles

Biocompatible nano- and microparticles are for example coated using the emulsion method and subsequent solvent evaporation (evaporation method).

Exemplarily, nano- and microparticles to be coated consist of a water-insoluble biocompatible polymer or a water-insoluble lipid and an active substance for pulmonary application. The polymer is for example a polyester, polyanhydride, polyorthoester, polyphosphoester, polycarbonate, polyketal, polyurea, polyurethane, block copolymer (PEG-PLGA), star polymer or comb polymer.

The water-insoluble lipid is for example chosen from the group of acylglycerols (mono-, di- or triglycerols). The active substance of said biocompatible nano- and microparticles is chosen from the group of appetite suppressants/antiadipose agents, acidosis therapeutics, analeptics/antihypoxaemic agents, analgesics, antirheumatics, anthelmintics, antiallergics, antianemics, antiarrhythmics, antibiotics, antiinfectives, antidementives, antidiabetics, antidotes, antiemetics, antivertigo agents, antiepileptics, antihemorrhagic agents, haemostatics, antihypertensives, antihypoglycemics, antihypotensives, anticoagulants, antimycotics, antiparasitic agents, antiphogisitics, antitussives, expectorants, antiarteriosclerotics, beta-receptor blockers, calcium channel blockers, inhibitors of the renin-angiotensin-aldosterone system, broncholytics, anti-asthma agents, cholagogics, bile duct therapeutics, cholinergics, corticoids, diagnostics and agents for diagnostic preliminaries, diuretics, circulation-promoting agents, anti-addiction agents, enzyme inhibitors, enzyme-activating or stimulating agents, enzyme deficiency correcting compounds, receptor antagonists, transport proteins, fibrinolytics, geriatric agents, gout agents, influenza drugs, colds and flu remedies, gynecologic agents, hepatics, hypnotics, sedatives, pituitary and hypothalamus hormones, regulatory peptides, hormones, peptide inhibitors, immunomodulators, cardiacs, coronary agents, laxants, lipid-reducing agents, local anaesthetics, neural therapeutic agents, gastric agents, migraine agents, mineral preparations, muscle relaxants, narcotics, neurotropic agents, osteoporosis remedies, calcium/calcium metabolism regulators, remedies for Parkinson's disease, psychopharmaceuticals, sinusitis agents, roborantia, thyroid therapeutics, serums, immunoglobulins, vaccines, antibodies, sexual hormones and their inhibitors, spasmolytics, anticholinergic agents, thrombocyte aggregation inhibitors, antituberculosis agents, urological agents, vein therapeutics, vitamins, cytostatics, antineoplastic agents, homeopathic remedies, vasoactive agents, gene therapeutics (DNA/RNA derivatives), transcription inhibitors, virostatic agents, nicotine, agents against erectile dysfunction, nitric oxide and/or nitric oxide-liberating substances.

In the sense of the present invention, also such particles are included as active substances which are for example utilized in diagnostic imaging techniques, but also for therapeutic purposes, e.g. in chemo- and radiotherapy and in hyperthermia therapy.

Alternatively, coated biocompatible nano- and microparticles are also prepared using nano-precipitation, salting-out, polymerization or spray drying. The preparation procedures mentioned herein are known to the expert in this field.

The coating of said biocompatible nano- and microparticles with a stabilizer is alternatively carried out by subsequent, non-covalent coating by means of mixing uncoated particles with the stabilizer or by gas phase-coating of particles using chemical vapor deposition or, respectively, by spraying of the particles or by covalent attachment of the stabilizer to uncoated particles or by co-electrospraying. These methods are known to the expert in this field.

If said biocompatible nano- and microparticles are prepared by subsequent non-covalent coating via mixing of uncoated particles with the stabilizer using the emulsion method, the polymer or lipid is initially dissolved in a solvent under addition of an active substance. The dispersed organic phase generated in this process is then transferred into a constant aqueous phase containing a stabilizer. After mixing of both phases and sonication with ultrasound, the organic phase containing the solvent is subsequently removed by evaporation and the biocompatible nano- and microparticles of this invention are obtained in suspension. Suitable solvents in which the polymer used according to the present invention is soluble to at least 0.1% (w/w) are for example, but not exhaustively, dichloromethane, chloroform, ethyl acetate, benzyl alcohol, methyl ethyl ketone, propylene carbonate. In a preferred embodiment, polyvinyl alcohol (PVA) is used as stabilizer.

In a preferred embodiment, biocompatible polymer between 1 and 100 g/l and stabilizer between 0.1 and 25 g/l is used for the preparation of said coated biocompatible nano- and microparticles of this invention. In a particularly preferred embodiment, the biocompatible polymer concentration is 50 g/l and the stabilizer concentration 10 g/l for the preparation.

Utilization of Coated Biocompatible Nano- and Microparticles of this Invention

Coated biocompatible nano- and microparticles of this invention are utilized for the fabrication of a pharmaceutical formulation suitable for pulmonary administration. The term biocompatibility thereby means compatibility for tissue and cells at the target site, e.g. the lung.

The stability of coated biocompatible nano- and microparticles of this invention is based on the prevention of particle aggregation during preparation, storage as well as before and during nebulization of said particles.

All features and advantages including the process steps illustrated in the claims, the description and the figures may be essential to the invention, either independently by themselves as well as combined with one another in any form.

### EMBODIMENTS

The following embodiment examples 1 and 2 describe the characterization of the stabilizer used for exemplarily coated biocompatible nano- and micro-particles, whereby the utilization of the stabilizer is not restricted to said particles. In the embodiments, polyvinyl alcohol (PVA) is exemplarily used as stabilizer and sildenafil as active substance. The coated biocompatible nano- and microparticles of the present invention are in the following also shortly referred to as particles. Poly(D,L-lactic-co-glycolide) copolymer (PLGA) is in the following shortly referred to as polymer.

### 1. Embodiment 1

### Preparation of Coated Biocompatible Nano- and Microparticles of this Invention Via Emulsion and Subsequent Evaporation

The procedure for the coating of biocompatible nano- and microparticles with a stabilizer is characterized by the following steps
a) dissolution of the biocompatible polymer or lipid and the active substance in a solvent under formation of an organic phase,
b) emulation of the organic phase in an aqueous phase containing a stabilizer,
c) mixture of the organic phase of step a) with the aqueous phase of step b),
d) removal of the solvent and obtaining the particles in suspension.

Coated biocompatible nano- and microparticles of this invention are for example synthesized at room temperature using the emulsion method with subsequent solvent evaporation known to those skilled in the art. For this purpose, between 1 and 100 g/l poly(D,L-lactide-co-glycolide) copolymer (PLGA) which is commercially available and can for example be obtained as Resomer® RG502H from Boehringer Ingelheim (Ingelheim, Germany) is initially dissolved with or without addition of between 1% and 20% of an active substance like for example the phosphodiesterase-5 inhibitor sildenafil, which is commercially available as free base and provided for example by AK Scientific (Mountain View, Calif., USA), in a water-immiscible solvent like for example methylene chloride. Then, 2 ml of the organic phase (dispersed phase) are transferred into 10 ml of an aqueous phase (constant phase) containing between 0.1 and 15 g/l of a surface stabilizer, for example polyvinyl alcohol (PVA). PVA is commercially available for example as Mowiol 4-88® provided by Sigma-Aldrich (Steinheim, Germany). After mixing both phases, the emulsion is sonicated. The organic phase is subsequently slowly removed by solvent evaporation in a rotary evaporator. Particles are utilized immediately after preparation.

### 2. Embodiment 2

### Characterization of Coated Biocompatible Nano- and Microparticles of this Invention

Coated biocompatible nano- and microparticles prepared according to embodiment 1 are characterized using methods and results as in the following described under embodiment 2, items 2.1 to 2.5. For this purpose, said coated biocompatible nano- and microparticles are either utilized directly after preparation or after nebulization with a nebulizer, for example Aeroneb® Professional provided by Aerogen (Dangan, Galway, Ireland) as specified by the manufacturer.

2.1 Diameter, Size Distribution and Surface Charge of Coated Biocompatible Nano- and Microparticles of this Invention

Freshly prepared coated biocompatible nano- and microparticles which are manufactured using the emulsion method with subsequent solvent evaporation as described in embodiment 1 are assessed in various combinations of polymer-concentration (ranging from 5 to 100 g/l) and PVA-concentration (ranging from 1 to 50 g/l) with respect to their properties diameter, size distribution and surface charge. Hydrodynamic diameter and size distribution (polydispersity PDI) of coated biocompatible nano- and microparticles is measured via dynamic light scattering (DLS). The zeta-potential as a measure for the surface charge is determined by laser Doppler anemometry (LDA), for example with a Zetasizer NanoZS/ZEN3600 (Malvern Instruments, Herrenberg, Germany). All measurements are performed at a temperature of 25° C. with aliquots appropriately diluted in filtrated and double-distilled water for DLS or with 1.56 nM NaCl for LDA, respectively. All measurements are carried out at least in triplicates with at least 10 repetitions immediately after preparation of the coated biocompatible nano- and micro-particles. In the following, n indicates the number of determinations.

A narrow particle size distribution, i.e. polydispersity indices (PDI) with a value below 0.1, is obtained with a PVA-concentration of more than 5 g/l at a constant PLGA concentration of 50 g/l or with a PLGA concentration between 10 and 50 g/l at a constant PVA-concentration of 10 g/l. FIG. 1 shows the size distribution which is determined by DLS of freshly prepared coated biocompatible nano- and microparticles. The mean size of coated biocompatible nano- and microparticles ranges from 100 to 400 nm (black line in FIG. 1). Coated biocompatible nano- and microparticles which are prepared using a PLGA concentration of 50 g/l and a PVA concentration of 10 g/l have a mean particle size of 195.1±9.6 nm (mean value±standard deviation, n=4), a narrow size distribution, i.e. a narrow polydispersity index (PDI) of 0.078±0.002 (mean value±standard deviation, n=4) as well as a negative surface charge, i.e. a negative zeta-potential of −5.7±0.8 mV (mean value±standard deviation, n=4).

To investigate diameter, size distribution and surface charge as a measure for the stability of coated biocompatible nano- and microparticles after nebulization, coated biocompatible nano- and microparticles of this invention are prepared with a theoretical content of 5% (w/w) of the active substance sildenafil (free base) according to embodiment 1 and characterized before and after nebulization with the nebulizer Aeroneb® Professional. For this purpose, nebulized suspensions of said coated biocompatible nano- and microparticles are collected and qualitatively analyzed as described in Dailey et al. (Dailey L A, Kleemann E, Wittmar M et al.: Surfactant-free, biodegradable nanoparticles for aerosol therapy based on the branched polyesters, DEAPA-PVAL-g-PLGA. Pharm. Res. 20(12), 2011-2020 (2003); Dailey L A, Schmehl T, Gessler T et al.: Nebulization of biodegradable nanoparticles: impact of nebulizer technology and nanoparticle characteristics on aerosol features. J. Controlled Release. 86(1), 131-144 (2003)). Suspensions of coated biocompatible nano- and microparticles are nebulized at an air flow rate of 5 l/min and collected by placing a glass microscope slide directly in front of the nebulizer T-shaped mouthpiece, which allows a deposition of aerosol droplets on the glass microscope slide. The resulting condensation fluid is collected for further analysis. The stability of nebulized biocompatible nano-polymer particles is assessed as described above using DLS.

Coated biocompatible nano- and microparticles of this invention have an average size of 197.1±1.7 nm, a narrow size distribution with a PDI of 0.074±0.005 as well as a negative surface charge with a zeta-potential of −5.1±0.3 mM. The parameters particle size, PDI and sildenafil content (see 2.3) are presented in FIG. 2 as quotient of values before and values after nebulization. As demonstrated in the figure, nebulization has no significant influence on the above mentioned parameters.

2.2 Stabilizer Layer Thickness of Coated Biocompatible Nano- and Microparticles of this Invention

The thickness of the adsorbed PVA layers serving as surface stabilizer of the coated biocompatible nano- and microparticles according to this invention is determined using DLS and zeta potential measurements as described under item 2.1 as a function of electrolyte concentration. Suitable determination methods are known to those skilled in the art. With respect to DLS measurements, the adsorbed PVA layer thickness (δ) is derived from comparing the particle size of bare (d₀) and coated (d_{ads}) biocompatible nano- and microparticles according to the following equation (1)

$$δ = \frac{{d}_{ads} - {d}_{0}}{2} \qquad ( 1 )$$

The layer thickness is calculated from zeta potential measurements using the Gouy-Chapman approximation known to the expert, which expresses the decrease of the electrostatic potential as a function of the distance from the surface in the following equation (2)

ψₓ=ψ₀·e^{-kx} (2)

whereby ψₓ is the potential at a distance x from the surface, ψ₀ is the surface potential and k⁻¹ is the Debye length. An increase of the electrolyte concentration (NaCl) decreases the Debye length. Zeta potentials are defined as the electrostatic potentials at the position of the slipping plane which are assumed to occur only outside of the fixed aqueous layer of a biocompatible nano- and microparticle. From equation (2) results equation (3)

ln ψₓ=ln ψ₀−k·x (3)

If zeta potentials (ψₓ) are measured in different concentrations of NaCl (0, 0.1, 0.2, 0.5, 1, 2 and 5 mM) and plotted against k equal to 3.33·c^{1/2}, where c is the molarity of electrolytes, the increase in concentration compensates for the thickness of adsorbed polymer layers.

FIG. 3 shows the thickness of adsorbed PVA layers on coated biocompatible nano- and microparticles for freshly prepared (white squares) and also nebulized particles (black squares). Depicted in FIG. 3A is the layer thickness as a function of the PVA concentration used. For freshly prepared as well as for nebulized particles, the layer thickness ranges between 10 and 20 nm. This result is also confirmed by transmission electron microscopic images. For this purpose, a copper grid (for example S160-3, Plano, Wetzlar, Germany) is coated with a thin layer of a diluted suspension of said coated biocompatible nano- and microparticles. Said coated biocompatible nano- and microparticles are then dried on the grid and investigated using a transmission electron microscope (TEM, for example JEM-3020 TEM, JEOL, Eching, Germany) at an acceleration voltage of 300 kV. FIG. 3D shows a representative TEM image of a coated biocompatible nano- and microparticle of this invention, in which the PVA layer (applied concentration during synthesis according to embodiment 1 of 10 g/l) is clearly visible. The zeta potential, i.e. the surface charge of said particles is negative for all NaCl-concentrations assessed (FIG. 3B). The straight line in FIG. 3C indicates the linear fit of experimental data.

2.3 Stability and Integrity of Coated Biocompatible Nano- and Microparticles of this Invention During Nebulization

To determine the stability of coated biocompatible nano- and microparticles of this invention, aliquots of particle solutions are nebulized at an air flow rate of 5 l/min with an Aeroneb® Professional nebulizer according to the manufacturer's instructions, and aerosol droplets are collected as condensation fluid on a glass microscope slide. The resulting condensation fluid is subsequently analyzed using dynamic light scattering (DLS) as described under item 2.1, and via electron microscopy.

FIGS. 4 A and B illustrate that particle size and polydispersity index are not influenced by nebulization. Furthermore, the integrity of particles is also maintained during nebulization (FIG. 4C).

2.4 Drug Content in Coated Biocompatible Nano- and Microparticles of this Invention

To determine the content of the active substance sildenafil in coated biocompatible nano- and microparticles of this invention prepared according to embodiment 1, for example 1 ml of a suspension of coated biocompatible nano- and microparticles is subjected to centrifugation at 16873×g for 30 min at 25° C. The supernatant is subsequently carefully removed and the amount of non-encapsulated active substance in the supernatant is determined. The pellets resulting from the centrifugation are freeze-dried, weighted and then dissolved in for example chloroform which is a suitable solvent for PLGA and sildenafil. The non-dissolved fraction (stabilizer) is removed by centrifugation. Subsequently, an aliquot is taken from the organic phase to assess the amount of encapsulated sildenafil. The sildenafil concentration is determined using UV/Vis spectroscopy with a spectrophotometer (for example Ultrospec® 3000, Pharmacia Biotech, Freiburg, Germany). The absorption all aliquots is measured at a wavelength of 291 nm. The active substance (AS) content of biocompatible nano- and microparticles (PLGA-BNP) is calculated with the aid of a calibration curve and defined in the following formula (4).

$$Ascontent ( \% ( w / w ) ) = \frac{massAS in PLGA - BNP}{massPLGA - BNP} \cdot 100 \qquad ( 4 )$$

In addition to the parameters particle size and PDI (see 2.1), the sildenafil content is depicted in FIG. 2 as quotient of value before and value after nebulization. The figure shows that nebulization has no significant influence on the sildenafil content.

2.5 Drug Release from Coated Biocompatible Nano- and Microparticles of this Invention

Investigations with respect to the in vitro release of the active substance sildenafil are carried out in phosphate-buffered saline at a pH value of for example 7.4 for 500 minutes at 37° C. These assays are performed with coated biocompatible nano- and microparticles having a theoretical active substance content of 5% (w/w). Aliquots of said coated biocompatible nano- and microparticle suspensions are transferred into glass tubes and diluted with medium consisting of phosphate-buffered saline (PBS) pH 7.4+0.1% sodium dodecyl sulfate (SDS). The subsequent incubation is performed at 37° C. with agitation of the aliquots. In parallel to the experimental assay, the active substance sildenafil is incubated alone in medium under identical conditions. Fractions are removed at pre-set time points and subjected to centrifugation.

The active substance sildenafil is in vitro released from coated biocompatible nano- and microparticles of this invention over a time period of up to 300 minutes (FIG. 5). The release from particles with polymer RG502H occurs over a time period of up to 90 minutes. During this time period, >95% sildenafil is continuously released from particles. Nebulization with Aeroneb® Professional has no influence on the release rate of sildenafil.

### FIGURE LEGENDS

FIG. 1

Size distribution of coated biocompatible nano- and microparticles of this invention, determined by dynamic light scattering (DLS). The black line indicates the density of particle sizes, the dashed line represents the cumulative distribution of particle sizes.

FIG. 2

Stability of coated biocompatible nano- and microparticles of this invention during nebulization with the nebulizer Aeroneb® Professional. The stability is shown as ratio of final to initial properties of particles of this invention (property_{f}/propertyᵢ) (A) (PDI=polydispersity index). Fractions of coated biocompatible nano- and microparticle suspensions are collected during nebulization to analyze the stability during the nebulization process. Values are given as the mean±standard deviation (n=4).

FIG. 3

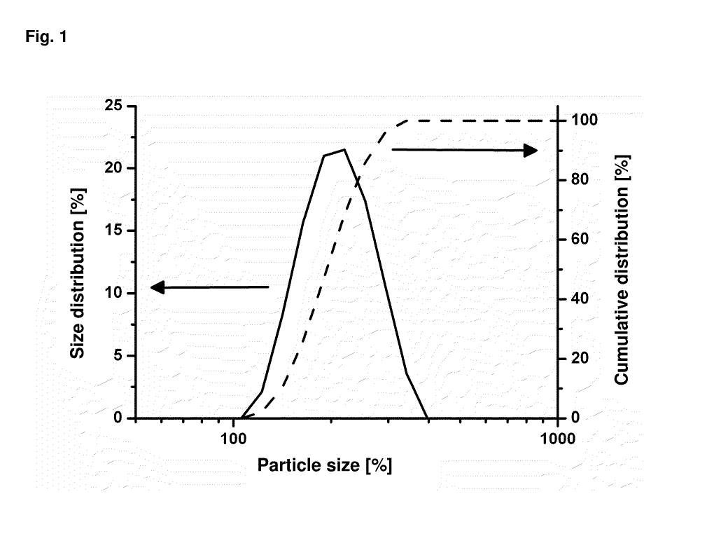

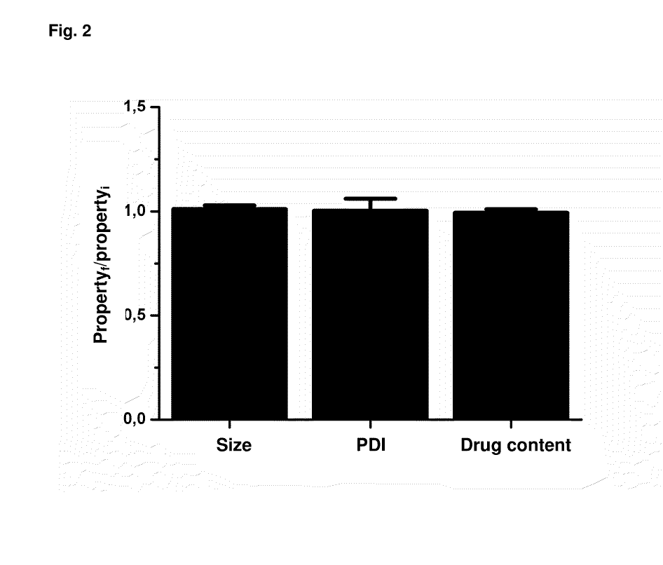

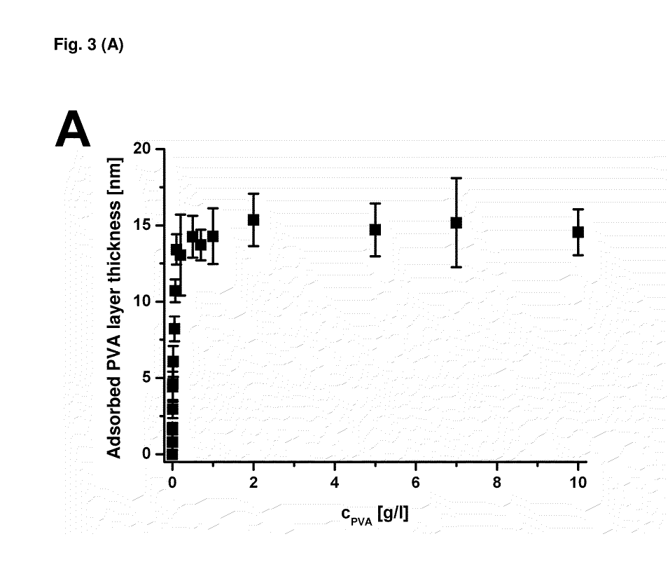

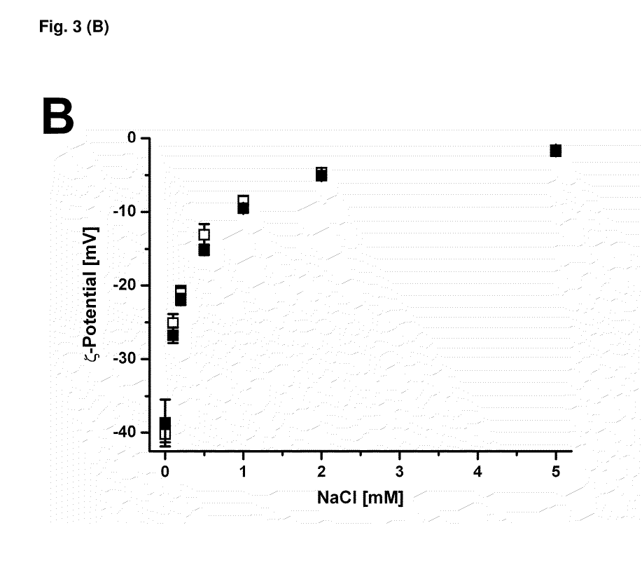

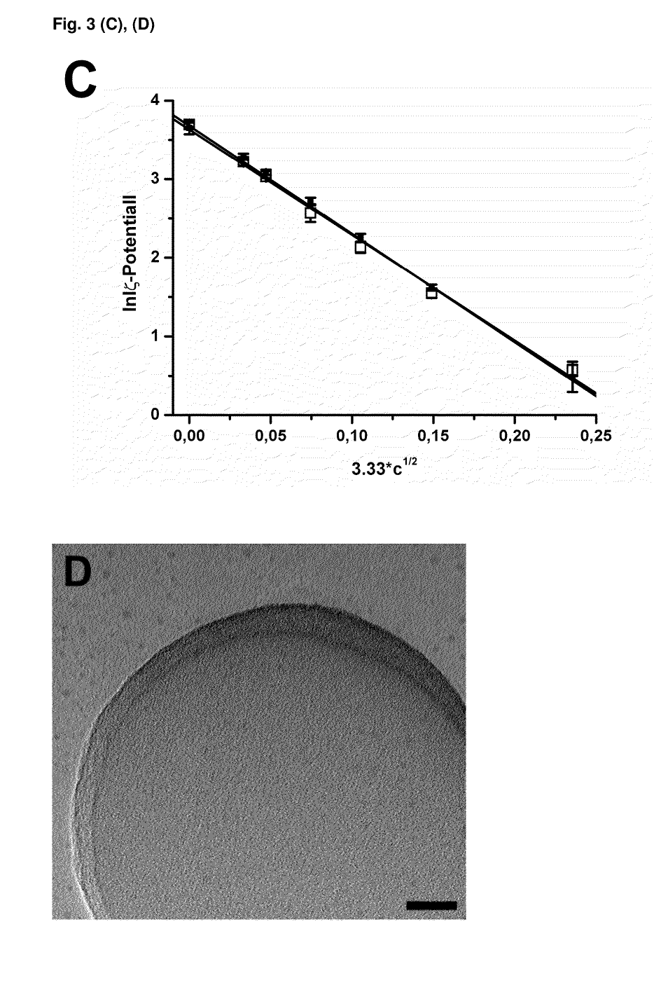

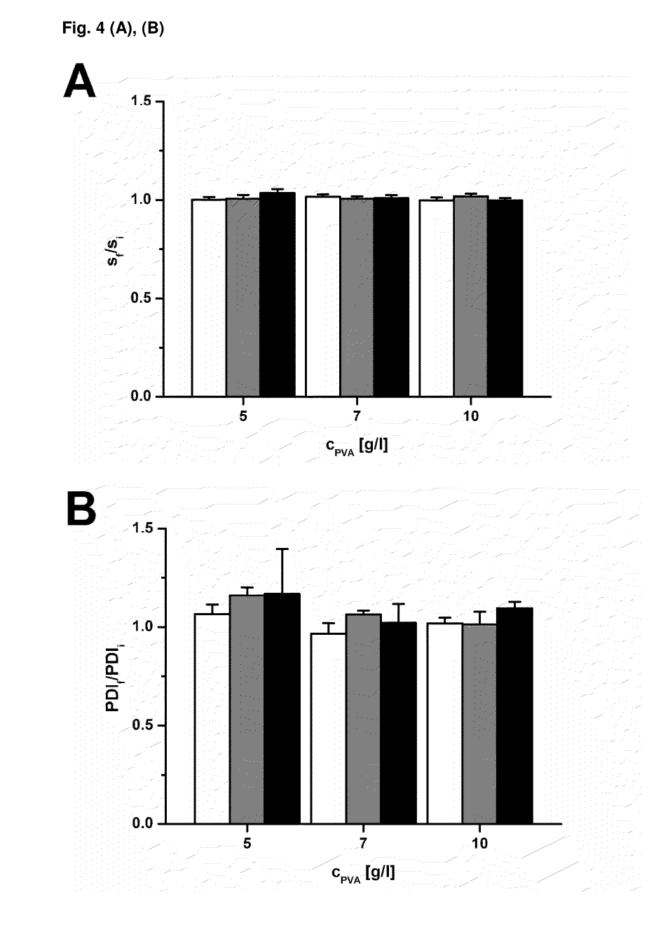

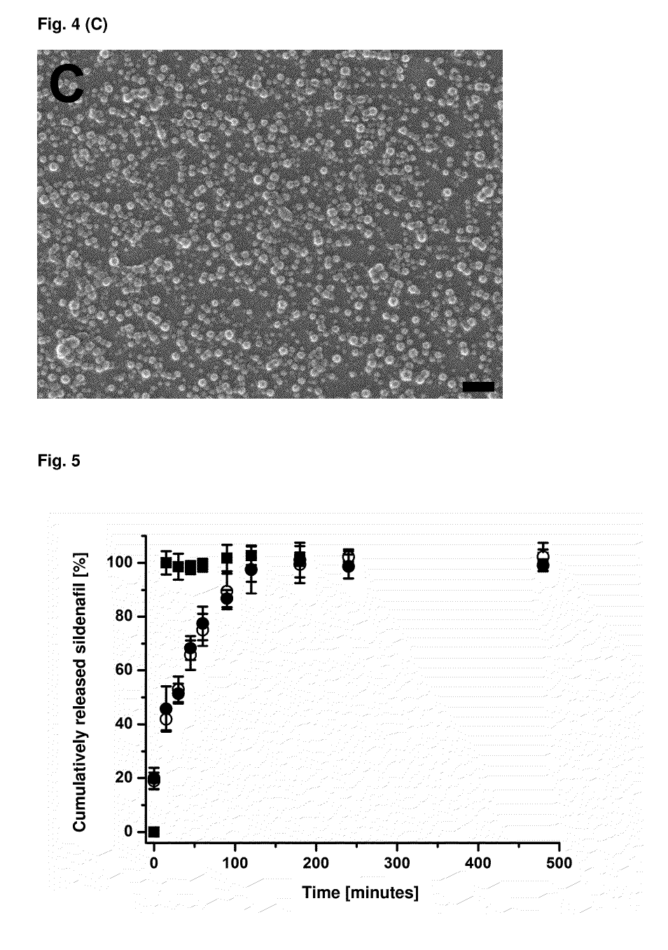

Adsorbed polyvinyl alcohol (PVA) layer thicknesses on coated biocompatible nano- and microparticles of this invention as a function of the PVA concentration (c_{PVA}) (A), and zeta potential of coated biocompatible nano- and microparticles prepared in PVA solution as a function of the electrolyte concentration (B). The slope (k) of the In|zeta potential| versus 3.33*c^{1/2} (concentration) gives the thickness of adsorbed polymer layers (C). White and black squares in (B) and (C) represent the properties of freshly prepared (B) or nebulized (C) biocompatible nano- and microparticles, respectively. The straight line in (C) represents the linear fit of experimental data (R²>0.99). The adsorbed PVA layer is clearly visible in the representative transmission electron microscopic image (D) (scale bar=20 nm). Values are given as the mean±standard deviation (n=4).

FIG. 4

Stability and integrity of coated biocompatible nano- and microparticles of this invention. Exemplarily shown is the stability of PLGA nanoparticles which were nebulized from a solution with various concentrations of the stabilizer PVA (c_{PVA}) using nebulizer Aeroneb® Professional according to the manufacturer's instructions. The stability is given as quotient of final (after nebulization) to initial (prior to nebulization) particle size (s_{f}/sᵢ) (FIG. 4A) and as (B) quotient of polydispersity indices (PDI) after (PDI_{f}) and before (PDIᵢ) nebulization (PDI_{f}/PDIᵢ) for different nanoparticle concentrations (white columns: 2 mg/ml; grey columns: 5 mg/ml; black columns: 10 mg/ml) (FIG. 4B). FIG. 4C shows a representative electron microscopic image of nanoparticles after nebulization (scale bar=1 μm). Values are given as the mean±standard deviation (n=4).

FIG. 5

In vitro sildenafil release profile from coated biocompatible nano- and microparticles of this invention (circles). White circles represent release characteristics of freshly prepared coated biocompatible nano- and microparticles, while black circles represent the release characteristics nebulized particles. Fractions of coated biocompatible nano- and microparticle suspensions are collected during nebulization to analyze the influence of nebulization on the sildenafil release profile from said coated biocompatible nano- and microparticles. Added for comparison is the dissolution profile of sildenafil as powder (black squares). Values are given as the mean±standard deviation (n=4).

1. A method of preparing a pharmaceutical agent for pulmonary application comprising the step of coating biocompatible nano- and microparticles with a stabilizer selected from the group of non-ionic surfactants, anionic surfactants, cationic surfactants, amphoteric surfactants, phospholipids or polymers, with the effect that aggregation of said biocompatible nano- and microparticles is prevented.

2. The method according to claim 1, characterized in that the polymer is polyvinyl alcohol (PVA).

3. The method according to claim 1, characterized in that said nano- and microparticles are composed of a biocompatible polymer or lipid and an active substance which is suitable for pulmonary application.

4. The method according to claim 3, characterized in that the biocompatible polymer is selected from the group consisting of a polyester, polyanhydride, polyorthoester, polyphosphoester, polycarbonate, polyketal, polyurea, polyurethane, block copolymer (PEG-PLGA), star polymer and comb polymer.

5. The method according to claim 3, characterized in that the biocompatible lipid is chosen from the group of acylglycerols.

6. The method according to claim 1, characterized in that the stabilizer coating said biocompatible nano- and microparticles has a stabilizer layer thickness between 1 and 200 nm.

7. A procedure for the coating of biocompatible nano- and microparticles with a stabilizer according to claim 1, characterized by the steps
e) dissolution of the biocompatible polymer or lipid and the active substance in a solvent under formation of an organic phase,
f) emulation of the organic phase in an aqueous phase containing a stabilizer,
g) mixing of the organic phase of step a) with the aqueous phase of step b),
h) removal of the solvent and obtaining the particles in suspension.

8. The procedure for the coating of biocompatible nano- and microparticles with a stabilizer according to claim 7, characterized in that the biocompatible polymer is selected from the group consisting of a polyester, polyanhydride, polyorthoester, polyphosphoester, polycarbonate, polyketal, polyurea, polyurethane, block copolymer (PEG-PLGA), star polymer and comb polymer.

9. The procedure for the coating of biocompatible nano- and microparticles with a stabilizer according to claim 7, characterized in that the biocompatible lipid is chosen from the group of acylglycerols.
